(19) United States
(12) Patent Application Publication
(10) Pub. No.: US 2006/0052286 A1
(43) Pub. Date: Mar. 9, 2006

(54) Factor VII conjugates for selectively treating neovascularization disorders

(75) Inventors: Alan Garen, New Haven, CT (US); Ron A. Adelman, Hamden, CT (US); Zhiwei Hu, New Haven, CT (US)

Correspondence Address: PATREA L. PABST;PABST PATENT GROUP LLP, 400 COLONY SQUARE, SUITE 1200, ATLANTA, GA, 30361, US

(73) Assignee: Yale University

(21) Appl. No.: 11/203,730

(22) Filed: Aug. 15, 2005

Related U.S. Application Data
(60) Provisional application No. 60/601,488, filed on Aug. 13, 2004.

(51) Int. Cl. A61K 38/41

(52) U.S. Cl. 514/6; 514/12; 530/383; 530/409

(57) ABSTRACT

Methods and compositions are provided for the treatment of diseases such as exudative macular degeneration, diabetic retinopathy, retinopathy of prematurity, choroidal neovascularization, retinal neovascularization, iris neovascularization, corneal neovascularization, ocular tumors, and other disorders of the eye, cancer, and inflammatory disorders. The method involves administering a conjugate, referred to as fVIIPD, containing a photosensitizer and a targeting molecule such as factor VII (“fVII”), fVIIa, or modified fVII, which binds with high affinity and specificity to tissue factor (TF). TF is more highly expressed, abnormally expressed or specifically expressed on endothelial cells lining the luminal surface of pathological neovasculature, than on normal vasculature, thus providing a specific and accessible therapeutic target. Following administration of fVIIPD, the compound specifically binds to the pathological neovasculature of the eye by interaction of the targeting molecule with TF expressed by endothelial cells within abnormal blood vessels. The photosensitizer may then be activated with a non-thermal laser light for selective destruction of abnormal vasculature.

### CROSS-REFERENCE TO RELATED APPLICATIONS

This application claims priority to U.S. Provisional Application No. 60/601,488 entitled “Neovascular Targeted Photodynamic Therapy” by Ron A. Adelman, Alan Garen, and Zhiwei Hu, filed Aug. 13, 2004.

### FIELD OF THE INVENTION

The present invention relates to the design, synthesis, and administration of reagents for treating patients with conjugates of coagulation factor VII and a photodynamic compound.

### BACKGROUND OF THE INVENTION

Medical science has recognized that angiogenesis is an important factor in the initiation and/or proliferation of a large number of diverse disease conditions. Angiogenesis is the physiological process involving the formation of new blood vessels from pre-existing vessels. Under normal physiological conditions, humans and other animals only undergo angiogenesis in very specific, restricted situations. For example, angiogenesis is normally observed in wound healing, fetal and embryonic development, and in the formation of the corpus luteum, endometrium and placenta. The process of angiogenesis has been found to be altered in a number of disease states, and in many instances, the pathological damage associated with the disease is related to uncontrolled angiogenesis. The diverse pathological states created due to unregulated angiogenesis have been grouped together as angiogenic dependent or angiogenic associated diseases. Therapies directed at control of the angiogenic processes could lead to the abrogation or mitigation of these diseases.

One example of a disease mediated by angiogenesis is ocular neovascular disease. This group of disorders is characterized by invasion of new blood vessels into the structures of the eye such as the retina, choroid, iris, or cornea. It is the most common cause of legal blindness in the United States and is involved in approximately twenty eye diseases. In Age-related Macular Degeneration (AMD), the associated visual problems are caused by an ingrowth of chorioidal vessels through defects in Bruch's membrane with proliferation of fibrovascular tissue beneath the retinal pigment epithelium.

Age-related Macular Degeneration (AMD) is the most common cause of legal, irreversible blindness in patients aged 65 and over in the US, Canada, England, Wales, Scotland and Australia. Although the average age of patients when they lose central vision in their first eye is about 65 years, some patients develop evidence of the disease in their fourth or fifth decade of life. Approximately 10% to 15% of patients manifest the exudative (wet) form of the disease. Exudative AMD is characterized by angiogenesis and the formation of pathological neovasculature. The disease is bilateral with accumulating chances of approximately 10% to 15% per annum of developing the blinding disorder in the fellow eye.

The hallmark of the exudative (wet) form of Age-Related Macular Degeneration is a Choroidal Neo-Vascular Membrane (CNVM) that grows beneath the retina or the RPE in the foveal-macular region. This CNVM leaks and bleeds evoking a scarring reaction that eventually results in the scarring of the affected area with consequential blindness. Histopathology of these CNVMs revealed that the vast majority of the membranes are nourished by a few (1 to 3) feeder vessels only. This means that only a few “vascular bridges” connect the origin of the CNVM (in the choroid) to the new location beneath the retina or the RPE.

One currently available treatment, as recommended by the Macular Photocoagulation Study (MPS), is photocoagulation of the membrane with an appropriate laser. Unfortunately, most of the membranes are sub-foveal when discovered and such a treatment modality leads to the complete destruction of all tissues, CNVM and retinal, within the treated area. It has been suggested that focusing on feeder vessel destruction would minimize the collateral damage caused by massive tissue ablation. The major problem with this feeder treatment is the limited patient eligibility because of the difficulty in identifying feeder vessels. (F Shiraga, et al. Ophthalmology, 105(4), 662-669, 1998.)

Common methods of treating abnormal vasculature use laser technology. One example of such methods used in the treatment of choroidal neovascularization (CNV), is photodynamic therapy (PDT). Photodynamic therapy for AMD involves injecting an intravenous photosensitizer and activating the photosensitizer with a non-thermal laser light. While this procedure is approved by the FDA, this method is not selective for abnormal blood vessels.

As a result, existing photodynamic therapies for the treatment of diseases characterized by pathological neovascularization are not satisfactory. The known techniques routinely damage blood vessels and tissues unrelated to the disease or the disease-causing areas.

Therefore, it is an object of the present invention to provide an improved treatment for disorders characterized by pathological neovascularization, including exudative macular degeneration, diabetic retinopathy, neovascular glaucoma, corneal neovascularization, and tumors.

### BRIEF SUMMARY OF THE INVENTION

Methods and compositions are provided for the treatment of diseases characterized by abnormal angiogenesis, such as exudative macular degeneration, diabetic retinopathy, neovascular glaucoma, choroidal neovascularization, retinal neovascularization, and other disorders of the eye, cancer, and inflammatory disorders. The method involves administering a conjugate, referred to as fVIIPD, containing a photosensitizer and a targeting molecule such as coagulation factor VII (“fVII”), fVIIa, or modified fVII, which binds with high affinity and specificity to tissue factor (TF). TF is more highly expressed, abnormally expressed or specifically expressed on endothelial cells lining the luminal surface of pathological neovasculature, than on normal vasculature, providing a specific and accessible therapeutic target. The targeting molecule is preferably factor VII (fVII) or the activated form factor VIIa (fVIIa)), which is the natural ligand for TF and one of the central proteins in the coagulation cascade. The fVIIPD specifically binds to the pathological neovasculature by the interaction of the targeting molecule with the tissue factor expressed by the endothelial cells within the abnormal blood vessels. The photosensitizer may then be activated with a non-thermal laser light for the selective destruction of abnormal vasculature.

The examples demonstrate the preparation of the conjugate and its efficacy in a rat model. CNV lesions were induced by laser photocoagulation of the retina in both eyes of Norway-Brown rats. Group 1: one eye received an intravitreal injection of 10 ul verteporfin conjugated to targeting molecule, and the other eye was used as a control. One hour later some of the lesions were treated with 690 nm laser light, while the other lesions were not irradiated. Group 2: one eye received iv. injection of 0.5 to 1 mg/m² verteporfin conjugated with targeting molecule. 15, 30 or 60 minutes later some of the lesions were treated with 690 nm laser light, while the other lesions were not irradiated. The lesions were examined by fluorescein angiography 1, 7 and 14 days later to assess the efficacy of targeted photodynamic therapy. In the case of intravitreal treatment with the same dosage, the effects of targeted photodynamic therapy lasted at least seven days. While there is no effect of PDT in the case of intravitreal injection of free verteporfin, the effects of targeted photodynamic therapy last even longer than 14 days, and the dosage of verteporfin in targeted photodynamic therapy is only 16% as compared to regular PDT verteporfin.

### DETAILED DESCRIPTION OF THE INVENTION

A method of treating disorders characterized by neovascularization through targeted photodynamic therapy has been developed. As described in more detail below, a photosensitizer is attached to a targeting molecule which selectively binds to endothelial cells of abnormal blood vessels. This allows for targeted photodynamic therapy such that primarily abnormal blood vessels are targeted and destroyed by the photodynamic therapy.

I. Compositions

The compositions are conjugates containing a photosensitizer covalently bonded to a targeting molecule capable of selectively binding to a cell surface marker. The conjugates may also contain a linker and/or tether molecule that serves to bind the photosensitizer compound to the targeting molecule.

A. Targeting Molecules (fVII)

The targeting molecule can be any polypeptide or protein that can selectively bind to a cell surface marker such as, for example, an extracellular region of surface bound tissue factor (TF).

As used herein the terms “polypeptide” and “protein” refer to a polymer of amino acids of three or more amino acids in a serial array, linked through peptide bonds. The term “polypeptide” includes proteins, protein fragments, protein analogues, and oligopeptides. The term “polypeptides” also contemplates polypeptides as defined above that are encoded by nucleic acids, produced through recombinant technology, isolated from an appropriate source, or are synthesized. The term “polypeptide” further contemplates polypeptides as defined above that include chemically modified amino acids or amino acids covalently or noncovalently linked to labeling ligands. “Truncated” as used herein refers to a polypeptide or protein that has fewer amino acids than a parent polypeptide or protein. The difference in the amino acid sequence may be at one or both of the termini of an amino acid sequence or due to amino acids deleted from the interior of the sequence when compared to the parent amino acid sequence.

The terms “cell surface antigen” and “cell surface marker” as used herein may be any antigenic structure on the surface of a cell. The cell surface antigen may be, but is not limited to, a tumor associated antigen, a growth factor receptor, a viral-encoded surface-expressed antigen, an antigen encoded by an oncogene product, a surface epitope, a membrane protein which mediates a classical or atypical multi-drug resistance, an antigen which mediates a tumorigenic phenotype, an antigen which mediates a metastatic phenotype, an antigen which suppresses a tumorigenic phenotype, an antigen which suppresses a metastatic phenotype, an antigen which is recognized by a specific immunological effector cell such as a T-cell, or an antigen that is recognized by a non-specific immunological effector cell such as a macrophage cell or a natural killer cell. Examples of “cell surface antigens” include, but are not limited to, CD5, CD30, CD34, CD45RO, CDw65, CD90 (Thy-1) antigen, CD117, CD38, and HLA-DR, AC133 defining a subset of CD34+ cells, CD19, CD20, CD24, CD10, CD13, CD33 and HLA-DR. Cell surface molecules include carbohydrates, proteins, lipoproteins or any other molecules or combinations thereof, that may be detected by selectively binding to a ligand or labeled molecule by methods such as, but not limited to, flow cytometry, FRIM, fluoresence microscopy and immunohistochemistry.

The term “tissue factor” as used herein refers to a transmembrane protein which complexes with coagulation factor VII (and the activated form factor VIIa (fVIIa)), and is the primary regulator of blood coagulation.

The term “fVII” refers to fVII, fVII mutated at one or more amino acids, preferably K341A or S344A, to not have coagulation activity but which still binds to tissue factor, and fVIIa. FVIIa typically refers to “single chain” coagulation factor VII. The term “factor VIIa”, or “fVIIa” means “two chain” activated coagulation factor VII cleaved by specific cleavage at the Arg 152-Ile153 peptide bond. FVII mutated refers to a truncated or modified form of fVII or fVIIa which does not have coagulation activity but which still binds tissue factor. Factor VIIa may be purified from blood or produced by recombinant means. The covalent bonding of the linker to the polypeptide may be to the uncleaved factor VII which may be subsequently cleaved between the 152-153 amino acid positions, or to the cleaved fVIIa.

Human purified factor VIIa is preferably made by DNA recombinant technology, e.g. as described by Hagen et al., Proc. Natl. Acad. Sci. USA 83: 2412-2416, (1986) or as described in European Patent No. 200.421. Factor VIIa produced by recombinant technology may be native factor VIIa or a more or less modified factor VIIa provided that such factor VIIa has substantially the same biological activity for blood coagulation as native factor VIIa. Such modified factor VIIa may be produced by modifying the nucleic acid sequence encoding factor VII either by altering the amino acid codons or by removal of some of the amino acid codons in the nucleic acid encoding the natural fVII by known means, e.g. by site-specific mutagenesis. A preferred polypeptide is a component polypeptide of fVII, cleaved between amino acid positions 152-153 such that the component polypeptide receiving the linker may comprise the amino acid sequence between positions 1 and 152, 153-408 or derivatives thereofl. In a preferred embodiment, the polypeptide is a modified fVII containing a lysine to alanine mutation at residue 341, which inhibits its clotting activity. Any truncation or amino acid substitution must not alter the ability of the modified fVII to selectively bind to tissue factor, or be capable of forming a covalent bond with a linker molecule having a chloromethylketone group thereon.

Purified Factor VII may also be produced by the methods described by Broze and Majerus, J. Biol. Chem. 255 (4): 1242-1247, (1980) and Hedner and Kisiel, J. Clin. Invest. 71: 1836-1841, (1983). These methods yield factor VII without detectable amounts of other blood coagulation factors. An even further purified factor VII preparation may be obtained by including an additional gel filtration as the final purification step. Factor VII can then be converted into activated fVIIa by known means, e.g. by cleavage with a serine esterase such as factor XIIa, IXa or Xa. Alternatively, as described by Bjoern et al. (Research Disclosure, 269 September 1986, pp. 564-565), factor VII may be activated by passing it through an ion-exchange chromatography column, such as Mono QR™ (Pharmacia Fine Chemicals).

B. Photosensitizers

Photosensitizers that can be conjugated to the targeting molecule include photodynamic dyes. The dye should be capable of causing damage to the targeted tissue after exposure to the appropriate type of radiation, e.g., light of a certain wavelength, typically between about 630 nm and about 750 nm. Any of a number of available photodynamic dyes can be used, such as those described in U.S. Pat. Nos. 6,693,093 and 6,443,976, which include hematoporphyrins, including derivatives thereof such as dihematoporphyrin ethers and dimer and trimers of hematoporphyrins (examples of which are described in U.S. Pat. Nos. 4,968,715 and 5,190,966), and improvements thereon, examples of the latter being described in U.S. Pat. Nos. 5,028,621, 4,866,168, 4,649,151 and 5,438,071; aminolevulinic acids (precursors to hematoporphyrin) as sources of photodynamic compounds, as described and exemplified in U.S. Pat. No. 5,079,262; porphyrins, including boronated porphyrin, benzoporphyrin, and derivatives thereof, and as further exemplified by the green porphyrins described in U.S. Pat. Nos. 4,883,790, 4,920,143, 5,095,030 and 5,171,749; merocyanines; porphycenes; porfimer sodium; verteporfin (Vysudine™, CIBA Vision); Photofrin II™; PH-10™; chlorins, as exemplified by meso-tetra(hydroxyphenyl)-chlorin and bacteriochlorins, the latter exemplified in U.S. Pat. Nos. 5,171,741, 5,173,504; zinc phthalocyanine, as described in U.S. Pat. No. 5,166,197; purpurins, such as tin ethyl etiopurpurin (SnET2™, Miravant); pheophorbides, examples of which are described in U.S. Pat. Nos. 5,198,460, 5,002,962 and 5,093,349; and monoclonal antibody-dye conjugates of each of the foregoing, and, optionally; mixtures of any or all of the foregoing.

In a preferred embodiment, the photosensitizer is verteporfin. Verteporfin, also known as benzoporphyrin derivative mono acid or BDP-MA, is a second generation photosensitizer. Second generation photosensitizers are characterized by an absorption band at wavelengths greater than 630 nm, high extinction coefficients, high yields of singlet oxygen up irradiation, and short half-lives in the body. Unlike first generation photosensitizers where energy is transferred via the transfer of a hydrogen or an electron, second generation photosensitizers transfer chemical energy to ground state (triplet) oxygen to form singlet oxygen, a reactive species capable of inducing cell death by damaging lipid membranes, proteins, and nucleic acids. Verteporfin is a chlorin-type molecule which exists as an equal mixture of regioisomers, each of which consists of a pair of enantiomers. The structures of the two regioisomers of verteporfin are shown below: 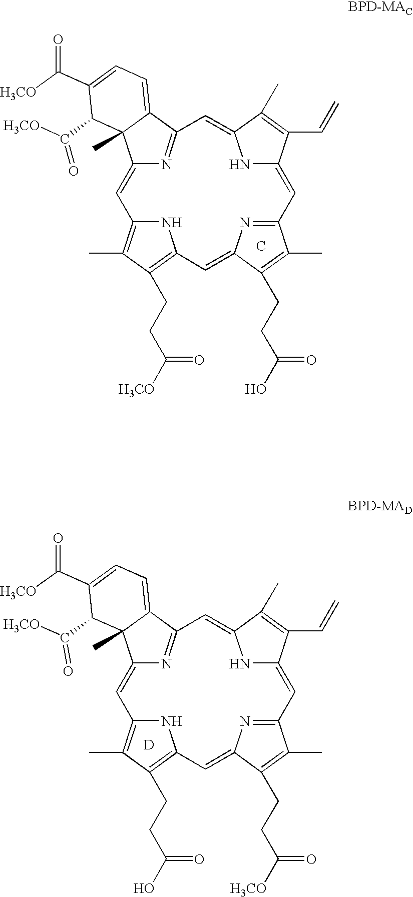
Verteporfin has an absorption spectrum with several peaks, including a strong absorption in the 680 -695 nm region. Verteporfin absorbs light efficiently at a wavelength of 689 nm (red light), which can penetrate a thin layer of blood, melanin, or fibrotic tissue. Verteporfin can be conjugated to the effector domain of fVII via amide bonds or ester bonds. For example, lysine residues in the effector domain can react with an ester group on verteporfin to form an amide.

Another patent that describes or identifies suitable photodynamic dyes is U.S. Pat. No. 5,910,510. Several photodynamic dyes are currently approved by the FDA, or are in clinical trials, for use in treating cancer. For example, Photofrin™ (porfimer sodium, manufactured by QLT PhotoTherapeutics and distributed by Sanofi Pharmaceuticals, Inc. and Beaufour Ipsen) is currently FDA-approved for early and late stage lung cancer and esophageal cancer. The following other photodynamic dyes are presently undergoing clinical trials and/or animal studies: Bopp™ (boronated porphyrin, Pacific Pharmaceuticals) for brain cancer, FoscanTM for head and neck cancer (Scotia QuantaNova), Lutrin.TM. for breast cancer (lutetium texaphyrin (Lu-Tex), Pharmacyclic) and PH-10 for cancerous tumors (Photogen Technologies, Inc.).

Although described above with reference specific to compounds, one can also utilize enantiomers, stereoisomers, metabolites, derivates and salts of the active compounds. Methods for synthesis of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid ; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa., 1985, p. 1418).

A prodrug is a covalently bonded substance which releases the active parent drug in vivo. Prodrugs are prepared by modifying functional groups present in the compound in such a way that the modifications are cleaved, either in routine manipulation or in vivo, to yield the parent compound. Prodrugs include compounds wherein the hydroxy or amino group is bonded to any group that, when the prodrug is administered to a mammalian subject, cleaves to form a free hydroxyl or free amino, respectively. Examples of prodrugs include, but are not limited to, acetate, formate and benzoate derivatives of alcohol and amine functional groups.

Metabolites result from biochemical processes by which living cells interact with the active parent drug or other formulas or compounds of the present invention in vivo. Metabolites include products or intermediates from any metabolic pathway.

c. Linkers
Linkers

The term “linker” as used herein refers a molecule capable of covalently connecting a cytotoxic compound to an amino acid side chain of a protein. The term “linker” may be a non-peptidyl linker or a peptidyl linker. The linker may have covalently bonded thereto a tether, as defined below, for covalently linking a cytotoxic compound to the linker. The term “peptidyl linker” as used herein refers to a peptide comprising at least two amino acids and which can be coupled to an amino acid side-chain of a protein. The linker may have a reactive group at the carboxyl terminus such as, but not limited to, a chloromethylketone. The peptide of the peptidyl linker may be cleavable by proteolytic enzymes found within a cell.

One suitable linker is a peptidyl methylketone linker covalently bonded to the polypeptide, most preferably to the side chain of an amino acid within the catalytic triad of the serine protease domain of fVIIa. In the human and bovine fVII proteins, the amino acids which form a catalytic “triad” are Ser344, Asp242, and His193. The catalytic sites in fVII from other mammalian species may be determined using presently available techniques including, among others, protein isolation and amino acid sequence analysis. Catalytic sites may also be determined by aligning a sequence with the sequence of other serine proteases, particularly chymotrypsin, whose active site has been previously determined by Sigler et al., J. Mol. Biol., 35:143-164 (1968), and determining from the alignment the analogous active site residues. Attachment of the peptidyl linker to this domain will inactivate the serine protease activity, thereby reducing the potential of the composition, when administered to an animal, to induce blood coagulation.

The terms “methylketone” and “chloromethylketone” as used herein refer to the carboxy terminus reactive moiety that may form the covalent bond between a peptide linker and an amino acid side chain of a recipient polypeptide. During the linkage reaction, the chloro group is removed. Thus, the unlinked peptidyl linker will have a chloromethylketone moiety and the covalently attached peptide will have a methylketone moiety without a halogen atom thereon.

Peptidyl linkers suitable for use, before being bonded to the polypeptide, have a carboxy-terminus chloromethylketone group that may react with a suitable amino acid side chain of the polypeptide. Preferably, but not necessarily, the carboxy terminal amino acid having the chloromethylketone group thereon is an arginine. Although any peptidyl chain sequence or length may be used, a suitable peptide is a tripeptide. Preferred peptidyl linkers include, but are not limited to, tyrosine-glycine-arginine-chloromethylketone (YGR-ck); phenylalanine-phenylalanine-arginine-chloromethylketone (FFR-ck), glutamine-glycine-arginine-chloromethylketone (QGR-ck), and glutamate-glcine-arginine chloromethylkefone (EGR-ck). A most preferred linker is FFR-ck.

It will be understood by those of skill in the art that upon covalently attaching the chloromethylketone to the recipient polypeptide, the chloro-moiety is displaced. Accordingly, the term “FFR-ck-VIIa”, for example, refers to FFR-methylketone tripeptidyl linker bonded to fVIIa and not having a chloro-atom attached thereto.

It is believed that a complex, formed from photosensitizer-phenylalanyl-phenylalanyl-arginyl-ck-VIIa (FFR-ck-VIIa) and tissue factor (TF) expressed on the plasma membrane of cells, may be internalized in a FFR-ck-VIIa concentration-dependent manner by ligand-receptor mediated endocytosis. The ligand-receptor complex is endocytosed into early and late endosomes and is delivered to lysosomal vesicles and degraded by lysosomal enzymes. Accordingly, the peptide selected for use as a linker peptide is also suitable for cleavage by an intracellular hydrolytic activity of the target cell enzyme. When so cleaved, after endocytotic internalization, the photosensitizer attached to the linker may be released from a polypeptide such as fVIIa. The released photosensitizer may then modulate a physiological function of the target cell.

A number of different linkers can be used. For example, linkers can be an arginyl methylketone such as phenylalanine-phenylalanine-arginine methylketone, tyrosine-glycine-arginine methylketone, glutamine-glycine-arginine methylketone, glutamate-glycine-arginine methylketone or phenylalanine-proline-arginine methylketone. In a preferred embodiment, the linker is phenylalanine-phenylalanine-arginine methylketone. In another preferred embodiment, the linker is tyrosine-glycine-arginine methylketone. In another preferred embodiment, a linker is covalently bonded to an amino acid side chain within a serine protease active site of fVIIa, thereby inactivating the serine protease active site.
Tethers

The term “tether” as used herein refers to a molecule that can form a hydrolysable bond such as, but not limited to, a carbamate, an amide, an ester, a carbonate or a sulfonate bond with a cytotoxic compound such as, but not limited to, a curcuminoid, and which can also be covalently bonded to a linker such as, but not limited to, the N-terminus of a linker, including a peptidyl linker, thereby connecting the cytotoxic compound to the linker via the tether. Suitable tethers include a dicarboxylic acid, a disulfonic acid, an omega-amino carboxylic acid, an omega-amino sulfonic acid, an omega-amino carboxysulfonic acid, or a derivative thereof, wherein the tether may comprise 2-6 carbons in any arrangement such as a linear, branched or cyclic carbon arrangement, and wherein the tether is capable of forming a hydrolysable bond. The photosensitizer may be covalently bonded to a tether, which preferably is a dicarboxylic acid or caproyl moiety. Another exemplary tether is succinate that may be bonded to a curcuminoid by the addition of succinic anhydride. The hydrolysable bond can be a carbamate, an amide, an ester, a carbonate and a sulfonate.

In one embodiment, the photodynamic compound is linked to the peptide via a tether that functions as a linker and as a tether. In a preferred embodiment, the photodynamic compound is connected to the peptide via both a tether and a linker. The tether forms a hydrolysable bond with the photosensitizer and is covalently bonded to a linker, which is covalently bonded to an amino acid side chain of the protein.

In yet another preferred embodiment, the photosensitizer compound is bonded to a tether, which is covalently linked to an N-terminal amino acid of a peptidyl linker such as phenylalanine-phenylalanine-arginine, the C-terminal amino acid of which comprises a methylketone. The methylketone group forms a covalent bond with an amino acid side group of fVIIa that does not prevent the conjugated construct from selectively binding to tissue factor expressed on a cell membrane.

In yet other embodiments, the tether can be a dicarboxylic acid, a disulfonic acid, an omega-amino carboxylic acid, an omega-amino sulfonic acid, an omega-amino carboxysulfonic acid, or a derivative thereof, wherein the tether comprises 2-6 carbons, and wherein the tether is capable of forming a hydrolysable bond. The tether can also be succinate.

II. Method of Making Targeting Molecule-Photosensitizer Conjugates

A. Conjugation

The compositions comprise one or more photosensitizers bound to a targeting molecule. The photosensitizer can be bound directly to the targeting molecule, for example, through the reaction of reactive functional groups on the photosensitizer and the targeting molecule. Alternatively, the photodynamic compound can be conjugated to the targeting molecule via a linker and/or tether molecule.

Porphyrins can be conjugated to targeting molecules, such as proteins, by the formation of ester or amide bonds. For example, verteporfin is reacted with dichloroethane (“EDC”) in the presence of N-hydroxysulfosuccinimide (“Sulfo-NHS”) and fVII to form the verteporfin-fVII conjugate as shown below. Sulfo-NHS can be used to aid in binding primary amines to carboxylic acid groups.
Dye+EDC, with or without Sulfo-NHS+fVIIP→Dye−(H—N)::::C═O-(fVIIP)
Porphyrins can also be conjugated to proteins using carbodiimide or N-hydroxysuccinimide ester (HNS)-based protocols as described in Hamblin et al., J. Photochem. Photobiol., B 26, 45-56 (1994); Mew et al., J. Innumol., 130, 1473-1477 (1983); Donald et al., Otolaryngol.-Head Neck Surg., 6, 781-787 (1991); Hamblin et al., Photochem. Photobiol., 72, 533-540 (2000); Del Governatore et al.; Br. J. Cancer, 82, 56-64 (2000); Schmidt et al., Br. J. Cancer, 75, 54-61 (1997). Sutton et al. describe a method of conjugating porphyrins, chlorins, and bacteriochlorins to protein by synthesizing porphyrins, chlorins, and bacteriochlorins bearing a single amine-reactive isothiocyanate group. Sutton et al., Bioconjugate Chemistry, 13, 249-263 (2002).
III. Methods of Treatment

A. Pharmaceutically Acceptable Formulations

Pharmaceutically acceptable compositions are provided that comprise a therapeutically-effective amount of conjugate, formulated together with one or more pharmaceutically acceptable carriers (additives) and/or diluents for use as a therapeutic agent for the treatment of a pathological condition of an animal or human such as a macular degeneration, diabetic retinopathy, cancer or other neovascular based disease. The phrase “therapeutically-effective amount” as used herein means that amount of a compound, material, or composition which is effective for producing a desired therapeutic effect against cancer or other pathological comprising neovascularization.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or an encapsulating material such as liposomes, polyethylene glycol (PEG), PEGylated liposomes, or particles, which is compatible with the other ingredients of the formulation and not injurious to the patient. The phrases “parenteral administration” and “administered parenterally” as used herein means modes of administration other than enteral and topical administration, usually by injection, and include, without limitation, intravenous, intramuscular, intravitreal, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's vascular system.

The compositions may be delivered to an animal or human by any of these routes, depending on the disorder to be treated. The preferred route of administration is intravenous injection so that the effective dose of the compound can be delivered via the vascular system. The dose may be delivered by subcutaneous injection, intraperitoneal injection, direct injection into the tissue to be treated or a proximal blood vessel feeding the tissue to be treated for reducing dilution of the effective therapeutic composition, and to achieve more rapid application of the composition to the vascular cells. The affinity of the targeting molecule (i.e. fVII) for the cell surface marker (i.e. tissue factor) will localize the effective dose of the therapeutic composition for selectively targeting abnormal vasculature.

As described in detail below, the pharmaceutical compositions may be specially formulated for administration in solid or liquid form, for example, a sterile solution or suspension. Conventional techniques for preparing pharmaceutical compositions which can be used are described in Remington's Pharmaceutical Sciences, 1985. Suitable pharmaceutical preparations are made by mixing the pharmaceutical composition, preferably in purified form, with suitable adjuvants and a suitable carrier or diluent. Suitable physiological acceptable carriers or diluents include sterile water and saline. Suitable adjuvants, in this regard, include calcium, proteins (e.g. albumins), or other inert peptides (e.g. glycylglycine) or amino acids (e.g. glycine, or histidine) to stabilise the purified fVIIa. Other physiological acceptable adjuvants are non-reducing sugars, cyclodextrins (cyclic carbohydrates derived from starch), polyalcohols (e.g. sorbitol, mannitol or glycerol), polysaccharides such as low molecular weight dextrins, detergents (e.g. polysorbate) and antioxidants (e.g. bisulfite and ascorbate). The adjuvants are generally present in a concentration of, but not limited to, from 0.001 to 4% w/v. The pharmaceutical preparation may also contain protease inhibitors, e.g. aprotinin, and preserving agents.

The preparations may be sterilized by, for example, filtration through a bacteria-retaining filter, by incorporating sterilizing agents into the compositions, by irradiating the compositions. They can also be manufactured in the form of sterile solid compositions which can be dissolved in sterile water, or some other sterile medium suitable for injection prior to or immediately before use.

Wetting agents, emulsifiers and lubricants, such as sodium lauryl sulfate and magnesium stearate, as well as coloring agents, release agents, coating agents, preservatives and antioxidants can also be present in the compositions.

Pharmaceutical compositions suitable for parenteral administration may comprise one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed include water, saline, balanced salt solution, ethanol, polyols (such as glycerol, propylene glycol, polyethylene glycol,), and suitable mixtures thereof. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid,. It may also be desirable to include isotonic agents, such as sugars or sodium chloride into the compositions. In addition, prolonged absorption of the injectable pharmaceutical form may be brought about by the inclusion of agents which delay absorption such as polyethylene glycol (PEG), aluminum monostearate and gelatin.

In some cases, in order to prolong the effect of a drug, it is desirable to slow the absorption of the drug from subcutaneous or intramuscular injection. This may be accomplished by coupling to PEG, the use of a liquid suspension of crystalline or amorphous material having poor water solubility. The rate of absorption of the drug then depends upon its rate of dissolution which, in turn, may depend upon size, form and amount of PEG, crystal size and crystalline form. Alternatively, delayed absorption of a parenterally-administered drug form is accomplished by dissolving or suspending the drug in an oil vehicle.

Injectable depot forms are made by forming microencapsuled matrices of the subject peptides or peptidomimetics in biodegradable polymers such as the polyhydroxy acids such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions are intended for parenteral, topical or local administration for prophylactic and/or therapeutic treatment. Most preferably, the pharmaceutical compositions are administered parenterally, i.e., intravenously, so that the compositions may be rapidly transported to a selected target cell such as a cancer cell or neovascular endothelial cell. Thus, compositions are provided for parenteral administration which comprise a solution of the fVIIPD dissolved in an acceptable carrier, preferably an aqueous carrier. A variety of aqueous carriers may be used, e.g., water, buffered water, 0.4% saline, 0.3% glycine. The fVIIPD can also be formulated into liposome preparations for delivery or targeting to sites of injury. The compositions may be sterilized by conventional, well known sterilization techniques. The resulting solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, for example, sodium acetate, sodium lactate, sodium chloride, potassium chloride, calcium chloride, etc.

B. Disorders to Be Treated

Disorders to be treated include any disorder characterized by uncontrolled or pathological angiogenesis, and preferably those characterized by overexpression of tissue factor. A switch from a quiescent state to an angiogenic state in the pathologic vasculature is usually activated by vascular endothelial growth factor (VEGF). One response activated by the binding of VEGF to receptors on vascular endothelial cells is the expression of tissue factor, a transmembrane receptor that binds plasma factor VII/VIIa to initiate blood coagulation. Because only the vascular endothelial cells that have bound VEGF express tissue factor, a putative target for the tumor vasculature is tissue factor expressed on endothelial cells which should bind factor VII/VIIa circulating in the blood. In addition to macular degeneration, this method should be useful for other neovascular disorders such as macular degeneration, diabetic retinopathy, retinopathy of prematurity, pathologic myopia, neovascular glaucoma, sickle cells disease, corneal neovascularization, angiomas, melanomas, carcinomas, sarcomas, and other solid tumors
1. Corneal Neovascularization

Neovascularization (NV) of the cornea represents a state of disease secondary to a variety of corneal insults, including contact lens (CL) wear. Diseases associated with corneal neovascularization that can be treated include but are not limited to, corneal graft rejection, , contact lens overwear, atopic keratitis, superior limbic keratitis, pterygium keratitis sicca, Sjogren's, acne rosacea, phylectenulosis, syphilis, Mycobacteria infections, lipid degeneration, chemical bums, bacterial ulcers, fungal ulcers, herpes simplex infections, herpes zoster infections, protozoan infections, trauma, rheumatoid arthritis, systemic lupus, polyarteritis, Wegener's sarcoidosis, scleritis, Stevens-Johnson disease, pemphigoid, radial keratotomy, and corneal graft rejection.

Conjunctival neovascularization that can be treated include but are not limited to pinguecula, pterygium, squamous cell carcinoma, pre-malignant lesions, scarring.
2. Skin and Eyelids

Skin and eyelid lesions associated with angiogenesis that can be treated include but are not limited to squamous cell carcinoma, basal cell carcinoma, angioma, hamangioma, scar, granuloma, other tumors of skin, eyelids, and orbit.
3. Retinal/Choroidal Neovascularization

With choroidal neovasculatization, abnormal blood vessels stemming from the choroid grow up through the retinal layers. Diseases associated with retinal/choroidal neovascularization that can be treated include, but are not limited to, diabetic retinopathy, macular degeneration, retinopathy of prematurity, sickle cell retinopathy, myopic degeneration, histoplasmosis, sarcoidosis, angioid streaks, syphilis, pseudoxanthoma elasticum, Paget's disease, vein occlusion, artery occlusion, carotid obstructive disease, chronic uveitis/vitritis, mycobacterial infections, Lyme disease, systemic lupus erythematosus, Eales' disease, choroidal angioma, retinal angioma, ocular melanoma, Behcet's disease, retinitis or choroiditis, presumed ocular histoplasmosis, Best's disease, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovascularization of the iris ), neovascularization of angle, neovascular galucoma and diseases caused by the abnormal proliferation of fibrovascular or fibrous tissue including all forms of proliferative vitreoretinopathy, whether or not associated with diabetes.
4. Hemangiomas

One of the most frequent angiogenic diseases of childhood is hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Therapy-resistant hemangiomas exist that cannot be treated with therapeutics currently in use.
5. Cancer and Benign Tumors

Angiogenesis is prominent in solid tumor formation and metastasis. Angiogeneic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors, and benign tumors such as carcinmoas, sarcomas, angiomas, and pyogenic granulomas.

Angiogenesis is important in two stages of tumor metastasis. The first stage where angiogenesis stimulation is important is in the vascularization of the tumor, which allows tumor cells to enter the blood stream and to circulate throughout the body. After the tumor cells have left the primary site, and have settled into the secondary, metastasis site, angiogenesis must occur before the new tumor can grow and expand. Therefore, prevention or control of angiogenesis could lead to the prevention of metastasis of tumors and possibly contain the neoplastic growth at the primary site.

C. Dosages

The regimen for any patient to be treated with a pharmaceutical composition should be determined by those skilled in the art. The daily dose to be administered in therapy can be determined by a physician and will depend on the treated organ, cause of neovascularization, particular compound employed, on the route of administration and on the weight and the condition of the patient.

The amount of fVIIPD that should be administered is that sufficient to provide an amount of photosensitizer which is capable, upon excitation, to damage the targeted tissue. This amount can vary widely depending upon the mode of administration and the formulation in which it is carried. As it is generally recognized that there is a relationship between the type of photosensitizer, the formulation, mode of administration, and dosage level, adjustment of these parameters to fit the particular combination to ensure delivery of an effective amount of the fVIIPD to the targeted tissue is possible.

For retinal and choroidal disorders like macular degeneration and diabetic retinopathy, the preferred route of treatment is by intravitreal injection or intravenous injection with subsequent activation with non-thermal laser. The route of administration is the same for iris disorders. Topical (preferred), intracular or intravenous injection can be used for administration to the cornea. Either topical or systemic administration can be used to treat eyelids and skin..

The dosage is adjusted based on the route of administration and the disease. The treatment may be repeated as needed. The pharmaceutical composition can be administered in a single dose, but it can also be given in multiple doses with intervals between successive doses depending on the dose given and the condition of the patient. The pharmaceutical composition may be administered intravenously or it may be administered by other routes.

The dosage of the compound will depend on the condition being treated, the particular compound, and other clinical factors such as weight and condition of the patient and the route of administration of the compound. For the treatment of retinal and choroidal neovascularization the compositions may be administered by intravitreal injection or intravenous injection. A slow release device may be used for slow release in the eye or other organs. For the treatment of skin disorders, the compositions can be administered systemically or topically. For treatment of certain disorders, the fVIIPD may be applied locally to treat cancer, angiomas, pre-malignant conditions and other diseases and conditions in which angiogenesis occurs. The fVIIPD is administered as required to alleviate the symptoms of the disorder. Assays can be performed to determine an effective amount of the agent, either in vitro and in vivo. Methods are known to those skilled in the art, and can be used to determine an effective dose of these and other agents for the treatment and prevention of diseases or other disorders as described herein.

After fVIIPD is administered, the application of dye excitation radiation, preferably via laser light, should be withheld until the targeted tissues selectively retain the dye, i.e, until the dye concentration in the target tissue is optimal. This will minimize damage to the non-targeted tissue. The optimum time following administration until the application of radiation to the treatment site will vary, and depends upon the mode of administration, the form of administration, and the nature of the targeted tissue should any radiation inadvertently be administered to non-targeted tissue. By way of example, if a choroidal neovascularization is the targeted tissue, the time between administration and application of radiation ranges from about 1 minute to about 2 hours, advantageously between about 15 to about 60 minutes, and preferably about 30 minutes.

The fluence and irradiation during the treatment with the radiation source can also vary, depending upon the type of tissue undergoing treatment, the depth of treatment desired in the targeted tissue, and the amount of overlying fluid or blood. Generally, however, the fluence will vary between about 50-200 joules/cm². The irradiance typically varies from about 150-900 mW/cm², with a range between about 150-600 mW/cm² being preferred. However, the use of a higher irradiance level may be preferable because higher levels shorten the treatment time.

C. Photodynamic Therapy

Preferably, electromagnetic radiation, such as from ultraviolet to visible and infra red light, is delivered after administration of the compositions and formulations. “Low-dose PDT”, refers to a total photodynamic therapy experience at substantially lower levels of intensity than that ordinarily employed. Generally, there are three significant variables: the concentration of the photosensitizing drug, the intensity of the radiation employed and the time of exposure to light, which determines the total amount of energy ultimately delivered to the target tissue. Generally, an increase in one of these factors permits a decrease in the others.

For example, if it is desired to irradiate only for a short period of time the energy of irradiation or the concentration of the drug may be increased. Conversely, if longer time periods of irradiation are permitted, lower irradiation intensities and lower drug concentrations are desirable. The use of low dose PDT offers an additional advantage in the form of reducing the likelihood of PDT side effects such as damage to unintended tissues or vessels. Additionally the targeting will increase the efficacy of the treatment by aiming at the abnormal blood vessels and preserving normal vessels.

It is understood that the manipulation of these parameters will vary according to the nature of the tissue being treated and the nature of the photosensitizer (PS) employed. However, in general, low-dose PDT employs combinations of the drug concentration, radiation intensity, and total energy values which are several fold lower than those conventionally used for destroying target tissues such as tumors and unwanted neovascularization. One measure may be the product of PS concentration (e.g., in ng/ml)×intensity (e.g., in mW/cm2)×time (e.g., in seconds). However, it is difficult to set absolute numbers for this product since there are constraints on each of the parameters individually. For example, if the intensity is too low, the PS will not be activated consistently; if the intensity is too high, hyperthermic and other damaging effects may occur. Additionally, in some instances, ambient or environmental light available at the target cell or tissue undergoing PDT may be sufficient in the absence of additional deliberate irradiation.

Similarly, PS concentrations cannot vary over any arbitrary range. There may also be constraints on the time during which radiation can be administered. Accordingly, the product of the foregoing equation is only a rough measure. However, this approach may provide a convenient index that can be adjusted according to the relative potency of the PS employed, and in general, an increase in intensity would permit a decrease in time of irradiation, and so forth.

The present invention will be further understood by reference to the following non-limiting examples.

### EXAMPLES

### Example 1

### Synthesis of fVII-Verteporfin Conjugate

Materials and Methods

Construction of plasmid containing the mfVIIP cDNAs. The plasmid vector encoding mfVIIP was constructed by amplifying the mouse factor VII cDNA with a K341 A mutation from a previously constructed plasmid vector described in U.S. Pat. No. 6,924,359. The mouse (“M”) fVIIP cDNA contains the coding sequence for mfVII, a BamHI site, ribonuclease S-peptide (wild-type or mutated) and 6 Histidines with a Hind III at the 5′-end and Not I at the 3′-end. The PCR amplified cDNAs are sequentially digested with Hind III and Not I and ligated into the Hind III and Not I digested-pcDNA3.1 (+). The sequences of the inserts in the plasmids were confirmed by sequencing. A hfVIIP cDNA also has been constructed with mutated human fVII in place of mouse fVII.

Production and purification of the mfVIIP protein. The mfVIIP plasmid was transfected into Chinese Hamster Ovary (CHO) cells using the Superfect reagent (Qiagen), and the cells were selected for tranfected colonies. The colony with the highest expression of mfVIIP protein was grown in CHO serum-free culture medium (Excel 301, JRH Biosciences) supplemented with 1 μg/ml vitamin K1 (Sigma), and the medium was collected every 3-4 days. The media were pooled and the protein was purified using Ni-NTA resin (Qiagen). The purified protein was dialyzed against PBS pH7.4 and concentrated to at least 1 mg/ml. The purified protein was stored at −20C for labeling reaction with the dyes.

Extraction of Pure Verteporfin Dye from Liposomal Visudyne and Conjugation with FVIIP Protein.

Verteporfin was extracted from liposomal Visudyne (QLT) by acidification with 6 M HCl and separation of organic (verteporfin, lower phase) and aqueous (liposome, upper phase) layers using CH₂Cl₂. After concentrating, verteporfin was further purified by silica gel chromatography using an eluting solvent CH₂Cl₂/methanol (3:1) and vacuum dried. For conjugation to the fVIIP protein, verteporfin was dissolved in DMF to a final concentration of 10 mg/ml. The verteporfin was activated by mixing 2 ul of verteporfin solution in DMF with 2 ul of a 25 mg/ml solution of EDC in DMF and adding 6 ul DMF. The mixture was incubated at room temperate for 30 min, followed by addition of 80 ul of fVIIP (1 mg/ml) was added to the activated verteporfin and incubated at RT for 1 hr. The verteporfin-mfVIIP conjugate (mfVIIPD) was separated from unconjugated verteporfin with a Sephadex G50 spin column. The mfVIIPD was scanned on a spectrophotometer from 200 nm to 800 nm to measure the protein absorbance at 280 nm and the verteporfin absorbance at 689 nm. For the control PBS was added instead of mfVIIP protein to the activated dye.

Results

The mfVIIPD molecule absorbed at 280 nm and 689 nm, whereas free verteporfin and mfVIIP protein absorbed at 689 nm or 280 nm, respectively. These data indicate that verteporfin was successfully conjugated to the mfVIIP protein.

The same procedure was tested for conjugation of the mfVIIP to another dye, Sn Chlorin e6 (SnCe6). The conjugation to SnCe6 also was successful.

### Example 2

### Binding of mfVIIP Protein and mfVIIPD (Conjugated to Verteporfin) to Human Tumor Cells Expressing TF.

mfVIIP was conjugated to verteporfin using the procedure described above. The binding activity of the conjugate to human breast cancer cells was tested by flow cytometry

The results indicate that the binding activity, presumably to Tissue Factor on the tumor cells, was identical for the conjugated and non-conjugated molecules.

### Example 3

### Injection of fVII-targeted Verteporfin Followed by Laser Activation Stops Blood Vessel Leakage in Rats

Tissue factor (TF), a transmembrane receptor, forms an exceptionally strong and specific complex with its ligand fVII as the initial step of the blood coagulation pathway. TF is not normally highly expressed on vascular endothelial cells but is expressed in a significantly higher amount on endothelial cells of new vessels such as tumor vasculature, probably induced by angiogenic factors such as Vascular Endothelial Growth Factor (VEGF). A photosensitizer is attached to a targeting molecule called fVII, which selectively binds to endothelial cells of abnormal blood vessels. The model for the targeting molecule is a Camelid IgG1 antibody, which is composed of two heavy chains without associated light chains; each heavy chain contains a V_{H} targeting domain conjugated directly to the hinge region of the Fc effector domain. The targeting molecules are composed of one or two chains, each chain containing a fVII targeting domain conjugated to the hinge region of an IgG1 Fc effector domain. The two fVII domains provide an avidity effect for binding to cells expressing multiple TF molecules, resulting in an affinity for TF that is significantly higher than the strong affinity of the monomeric fVII molecule. The Fc domain can activate a powerful cytolytic attack against cells that bind the targeting molecule, mediated by components of the immune system containing Fc receptors, such as natural killer (NK) cells and the C1q protein that initiates the complement pathway.

The efficacy of the fVII/Fc for cancer immunotherapy was demonstrated in a mouse model of human cancer, using an adenoviral vector encoding the fVII as the delivery system (Hu, et al., PNAS, 96:8161-8166 (1999) and Hu and Garen, PNAS 97:9221-9225 (2000)). Clinical trials of the protocol are being arranged for melanoma and prostate cancer. The fVII/Fc shows remarkable specificity for binding to TF on endothelial cells of growing blood vessels induced by exposing the retina of the experimental animals to a laser beam. The experiments have demonstrated the efficacy of FVIIPD-Targeted Photodynamic Therapy ITPT) of choroidal neovascularization (CNV) in a rat model of age-related macular degeneration (ARMD).

Methods

CNV lesions were induced by laser photocoagulation of the retina in both eyes of Norway-Brown rats. Choroidal neovascularization was induced by photocoagulating the posterior pole of rats with argon green laser using a slit lamp and a fundus contact lens to deliver 200-700 mW power for 0.1 second to a 50 μm spot. The power was adjusted to produce a blister or small hemorrhage. If no bleeding was observed, an additional laser-induced lesion was produced next to the first spot. Neovascular membranes usually develop within 2 weeks.

After 4 weeks, FVII-verteporfin conjugate (fVIIPD) at doses of 0.5 or 1 mg/m² was injected intravenously into the tail vein of the rats. Some of the CNV lesions were irradiated with 689 nm laser for 83 seconds at 15, 30 or 60 minutes after intravenous injection, while the other lesions were not irradiated (no light controls). Control rats were intravenously injected with Verteporfin (6.0 mg/m2) and laser irradiation was done 15 minutes later. The lesions were examined by fundus photographs and fluorescein angiography 1, 7 and 14 days later to assess the efficacy of ITPT. Choroidal veovascular membranes were treated by intravitreal injection of Verteporfin attached to targeting molecules that bind selectively to the endothelial cells of pathologic vessels. The photosensitizer was activated with an opal laser. A similar experiment was performed by intravitreal injection of fVII-targeted verteporfin.

Results

Intravitreal injection of fVII-targeted verteporfin followed by laser activation completely stopped blood vessel leakage at 7 days. This method selectively destroys abnormal vessels, to which the photosensitizer is attached, while leaving neighboring tissues intact. The advantage of targeted photodynamic therapy compared to conventional photodynamic therapy is the fact that it is selectively targeted toward abnormal blood vessels, thus it combines the benefits of targeting with photodynamic therapy. Targeted photodynamic therapy increases the efficacy of conventional photodynamic therapy and decreases its side effects, since the normal blood vessels will be relatively preserved.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

We claim:

1. A conjugate comprising a photosensitizer conjugated to a targeting molecule wherein the conjugate selectively targets a molecule expressed at a higher level on endothelial cells lining the luminal surface of pathological neovasculature, as compared to normal vasculature.

2. The conjugate of claim 1 wherein the photosensitizer is a porphyrin.

3. The conjugate of claim 1 wherein the targeting molecule selectively binds to tissue factor.

4. The conjugate of claim 3 wherein the targeting molecule is coagulation factor VII or a derivative thereof which does not have coagulation activity but which binds to tissue factor.

5. The conjugate of claim 1 wherein the photosensitizer is conjugated to the targeting molecule by a peptide linker.

6. The conjugate of claim 1 further comprising a pharmaceutically acceptable carrier for intravenous or intravitreous administration.

7. A method of treating a disorder characterized by uncontrolled or pathological angiogenesis comprising administering a conjugate comprising a photosensitizer conjugated to a targeting molecule, wherein the conjugate selectively targets a molecule expressed at a higher level on endothelial cells lining the luminal surface of pathological neovasculature, as compared to normal vasculature, in a pharmaceutically acceptable carrier for intravenous or intravitreous administration, and exposing the conjugate to a laser causing photoexcitation in an amount effective to kill cells at the site where the conjugate binds.

8. The method of claim 7 wherein the disorder is characterized by overexpression of tissue factor.

9. The method of claim 7 wherein the disorder is selected from the group consisting of ocular neovascular disorders, macular degeneration, diabetic retinopathy, branch retinal vein occlusion, central retinal vein occlusion, branch retinal artery occlusion, branch retinal vein occlusion, retinopathy of prematurity, pathologic myopia, neovascular glaucoma, sickle cell disease, Eales' disease, corneal neovascularization, angiomas, melanomas, carcinomas, sarcomas, other solid tumors, and other neovascular disorders.

10. The method of claim 7 wherein the disorder is corneal or iris neovascularization and is selected from the group consisting of corneal graft rejection, neovascular glaucoma, rubeosis, contact lens overwear, atopic keratitis, superior limbic keratitis, pterygium keratitis sicca, and Sjogren's radial keratotomy, and corneal graft rejection.

11. The method of claim 7 wherein the disorder is conjunctival neovascularization, tumors, angiomas, pterygium, and pinguecula.

12. The method of claim 7 wherein the disorder is associated with Retinal/Choroidal Neovascularization and is selected from the group consisting of diabetic retinopathy, macular degeneration, retinopathy of prematurity, type 1 subretinal neovascularization, type 2 subretinal neovascularization, sickle cell retinopathy, myopic degeneration, histoplasmosis, sarcoidosis, angioid streaks, syphilis, pseudoxanthoma elasticum, Paget's disease, branch retinal vein occlusion, central retinal vein occlusion, branch retinal artery occlusion, central retinal artery occlusion, carotid obstructive disease, retinal angiomatous proliferation, occult choroidal neovascularization, minimally classic choroidal neovascularization, classic choroidal neovascularization, chronic uveitis/vitritis, mycobacterial infections, fungal infections, , systemic lupus erythematosus, Eales' disease, choroidal angioma, retinal angioma, Von Hippel Lindau disease, ocular melanoma, Behcet's disease, retinitis or choroiditis, presumed ocular histoplasmosis, Best's disease, optic pits, Stargardt's disease, pars planitis, chronic retinal detachment, hyperviscosity syndromes, toxoplasmosis, trauma and post-laser complications, diseases associated with rubeosis (neovascularization of the iris), neovascularization of angle, neovascular galucoma and diseases caused by the abnormal proliferation of fibrovascular or fibrous tissue including all forms of proliferative retinopathy, whether or not associated with diabetes.

13. The method of claim 7 wherein the disorder results from cutaneous, subcutaneous, or mucosal disorders selected from the group consisting of benign and malignant neoplasms, tumors, angiomas, carcinomas, melanomas, sarcomas, inflammatory disorders, pingecula, and pterygium.

14. The method of claim 7 for the treatment of chronic inflammation, hemangiomas, and cancers.

15. The method of claim 7 wherein the conjugate is administered by injection or intravitrously and the conjugate is exposed to laser light at a time between 15 minutes and four hours following administration.

16. The method of claim 7 wherein the photosensitizer is a porphyrin.

17. The method of claim 7 wherein the targeting molecule selectively binds to tissue factor.

18. The method of claim 17 wherein the targeting molecule is coagulation factor VII, VIIa, or a derivative thereof which does not have coagulation activity but which binds to tissue factor.

19. The method of claim 7 wherein the photosensitizer is conjugated to the targeting molecule by a peptide linker.
